(19) United States
(12) Patent Application Publication
(10) Pub. No.: US 2026/0000340 A1
(43) Pub. Date: Jan. 1, 2026

(54) REDUCED BCI VISIBILITY USING DICHOPTIC STIMULATION

(71) Applicant: Snap Inc., Santa Monica, CA (US)

(72) Inventors: Bertrand Oustrière, Paris (FR); Guillaume Ployart, Paris (FR); Nelson Steinmetz, Paris (FR); Sid Kouider, Paris (FR)

(21) Appl. No.: 18/759,400

(22) Filed: Jun. 28, 2024

(51) Int. Cl. A61B 5/378; G06F 3/01

(52) U.S. Cl. CPC A61B 5/378; G06F 3/015

(57) ABSTRACT

A dichoptic visual stimulus is presented to a user's eyes by presenting a first visual stimulus to a first eye of the user, the first visual stimulus being presented with a first modulation, and presenting a second visual stimulus to a second eye of the user, the second visual stimulus being presented without the first modulation. The first visual stimulus and second visual stimulus are configured to cause the user to perceive the first visual stimulus and the second visual stimulus as a single visual element, and to perceive the visual element as being unmodulated by the first modulation. Neural signals are obtained from a neural signal capture device configured to detect neural activity of the user. In response to determining, based on the neural signals, that the first eye is focused on the first visual stimulus, a computing system executes a command associated with the dichoptic visual stimulus.

### TECHNICAL FIELD OF THE INVENTION

The present disclosure relates to the operation of brain-computer interfaces involving visual sensing, and in particular to brain-computer interfaces using dichoptic visual stimuli that differ between the left and right eye views.

### BACKGROUND

In visual brain-computer interfaces (BCIs), neural responses to a target stimulus, generally among a plurality of generated visual stimuli presented to the user, are used to infer (or “decode”) which stimulus is essentially the object of focus at any given time. The object of focus can then be associated with a user-selectable or controllable action.

Neural responses may be obtained using a variety of known techniques. One convenient method relies upon surface electroencephalography (EEG), which is non-invasive, has fine-grained temporal resolution and is based on well-understood empirical foundations. Surface EEG makes it possible to measure the variations of diffuse electric potentials on the surface of the skull (i.e. the scalp) of a subject in real-time. These variations of electrical potentials are commonly referred to as electroencephalographic signals or EEG signals.

### BRIEF DESCRIPTION OF THE SEVERAL VIEWS OF THE DRAWINGS

To easily identify the discussion of any particular element or act, the most significant digit or digits in a reference number may refer to the figure number in which that element is first introduced.

FIG. 1 illustrates various examples of display device suitable for use with systems and methods discussed herein, in accordance with some examples.

FIG. 2 is a perspective view of a head-worn device, in accordance with some examples.

FIG. 3 illustrates a further view of the head-worn device of FIG. 2, in accordance with some examples.

FIG. 4A is a rear view of the eye-facing sides of a left near-eye display presenting a first visual stimulus in a first state and a right near-eye display presenting a second visual stimulus, in accordance with some examples.

FIG. 4B is the view of FIG. 4A of the left near-eye display presenting a first visual stimulus in a second state and the right near-eye display presenting the second visual stimulus, in accordance with some examples.

FIG. 5 is a schematic view of a dichoptic visual stimulus presented to left and right eyes of a user, showing the user's visual perception of a visual element corresponding to the dichoptic visual stimulus, in accordance with some examples.

FIG. 6 illustrates an electronic architecture for receiving and processing EEG signals, in accordance with some examples.

FIG. 7 illustrates a computing system incorporating a brain computer interface (BCI), in accordance with some examples.

FIG. 8 illustrates a user focusing on an object while the screen objects, including the focus object, all blink at different rates and times, in accordance with some examples.

FIG. 9 is an example of a blink control pattern in which the objects are each given blinking patterns that are distinctive in terms of start time, frequency and duty cycle, in accordance with some examples.

FIG. 10 illustrates screen display objects and filtered versions thereof providing an HSF version, an LSF version, and a modulated version, in accordance with some examples.

FIG. 11 illustrates a screen display object and a texture, as well as a modulated version combining the screen display object and the texture according to a temporal modulation scheme, in accordance with some examples.

FIG. 12 is a block diagram showing a software architecture within which the present disclosure may be implemented, in accordance with some examples.

FIG. 13 is a diagrammatic representation of a machine, in the form of a computer system within which a set of instructions may be executed for causing the machine to perform any one or more of the methodologies discussed, in accordance with some examples.

FIG. 14 is a flowchart illustrating operations of a method for visual BCI using dichoptic stimulation, in accordance with some examples.

### DETAILED DESCRIPTION

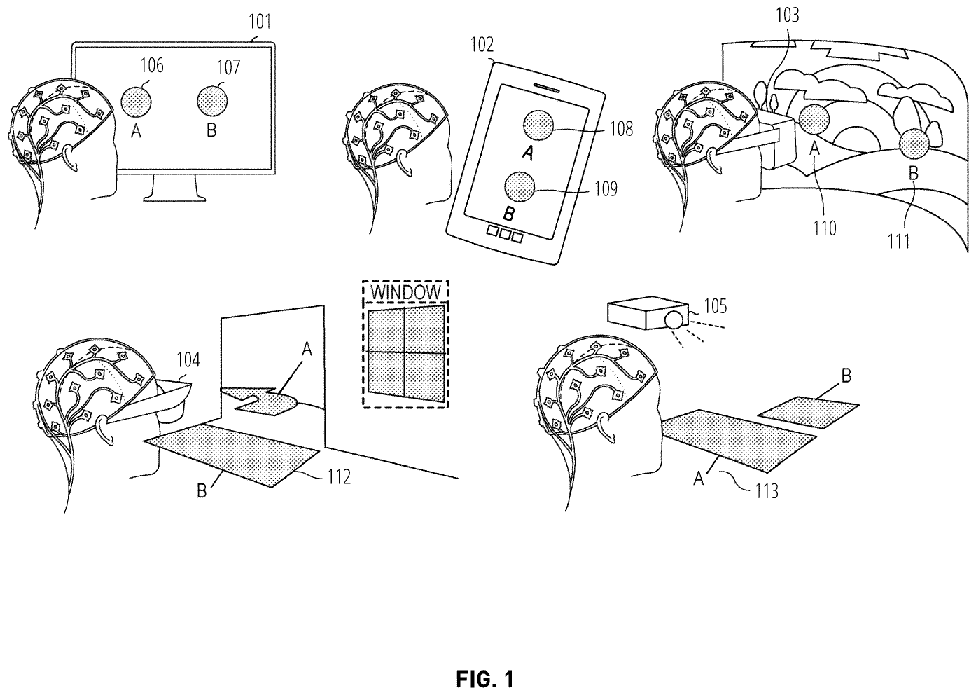

In a typical BCI, visual stimuli are presented by a display of a display device. Examples of suitable display devices (some of which are illustrated in FIG. 1) include television screens, computer monitors 101, projectors 105, virtual reality headsets 103, interactive whiteboards, and the display screens of tablets 102, smartphones, smart glasses 104, etc. The visual stimuli 106, 107, 108, 109, 110, 111, 112, and/or 113 may form part of a generated graphical user interface (GUI) or they may be presented as augmented reality or mixed reality graphical objects overlaying a base image: this base image may simply be the actual field of view of the user (as in the case of a mixed reality display function projected onto the otherwise transparent display of a set of smart glasses) or a digital image corresponding to the user's field of view but captured in real time by an optical capture device (which may in turn capture an image corresponding to the user's field of view amongst other possible views).

Some display devices provide binocular (or stereoscopic) imaging, such that three-dimensional virtual objects can be displayed. Binocular, stereoscopic, or three-dimensional display devices can include holographic displays, binocular head-mounted displays (HMDs), and so on. For example, a head-worn device may be implemented with a transparent or semi-transparent display through which a user of the head-worn device can view the surrounding environment. Such devices enable a user to see through the transparent or semi-transparent display to view the surrounding environment, and to also see objects or other content (e.g., virtual objects such as 3D renderings, images, video, text, and so forth) that are generated for display to appear as a part of, and/or overlaid upon, the surrounding environment (referred to collectively as “virtual content”). This is typically referred to as “extended reality” or “XR”, and it encompasses techniques such as augmented reality (AR), virtual reality (VR), and mixed reality (MR). Each of these technologies combines aspects of the physical world with virtual content presented to a user.

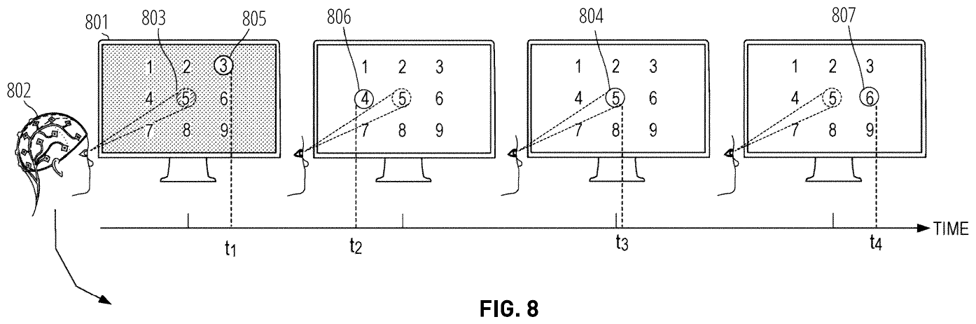

In a BCI, inferring which of a plurality of visual stimuli (if any) is the object of focus at any given time is fraught with difficulty. For example, when a user is facing multiple stimuli, such as for instance the digits displayed on an on-screen keypad (as shown in FIG. 8), it has proven nearly impossible to infer which one is under focus directly from brain activity at a given time. The user perceives the digit under focus, e.g., the digit “5”, meaning that the brain must contain information that distinguishes that digit from others, but current methods are unable to extract that information. Specifically, current methods can, with difficulty, infer that a stimulus has been perceived, but they cannot determine which specific stimulus is under focus using brain activity alone.

To overcome this issue and to provide sufficient contrast between stimulus and background (and between stimuli), the stimuli used by visual BCIs can be configured to blink or pulse (e.g. large surfaces of pixels switching from black to white and vice-versa), so that each stimulus has a distinguishable characteristic profile over time. The flickering stimuli give rise to measurable electrical responses. Specific techniques monitor different electrical responses, for example steady state visual evoked potentials (SSVEPs) and P-300 event related potentials. In some implementations, the stimuli flicker at a rate exceeding 6 Hz. As a result, such visual BCIs rely on an approach that consists of displaying the various stimuli discretely rather than constantly, and typically at different points in time. Brain activity associated with attention focused on a given stimulus is found to correspond (i.e. correlate) with one or more aspects of the temporal profile of that stimulus, for instance the frequency of the stimulus blink and/or the duty cycle over which the stimulus alternates between a blinking state and a quiescent state.

Thus, decoding of neural signals relies on the fact that when a stimulus is turned on, it will trigger a characteristic pattern of neural responses in the brain that can be determined from electrical signals, e.g., the SSVEPs, picked up by electrodes of an EEG device, such as the electrodes of an EEG helmet. This neural data pattern might be very similar or even identical for the various digits, but it is time-locked to the digit being perceived: only one digit may pulse at any one time so that the correlation with a pulsed neural response and a time at which that digit pulses may be determined as an indication that that digit is the object of focus. By displaying each digit at different points in time, turning that digit on and off at different rates, applying different duty cycles, and/or simply applying the stimulus at different points in time (all referred to collectively as “temporal modulation”), the BCI algorithm can establish which stimulus, when turned on, is most likely to be triggering a given neural response, thereby allowing a system to determine the target under focus.

Thus, some implementations of visual BCI rely on the temporal modulation (e.g., blinking) of visual stimuli capable of creating a strong EEG response in the user's brain. These EEG responses may be referred to as Event Related Potentials (ERPs). However, temporal modulation of visual stimuli for use by a BCI may introduce certain challenges to the presentation of visual content on a display. First, the blinking or otherwise temporally modulated visual stimuli are typically highly visible to the user. The user tends to be fully conscious of the temporal modulation, and can be annoyed or even disturbed by the modulation. Furthermore, because temporal modulation of visual stimuli often introduces specific constraints on the texture or other visual component of the visual stimulus that is being modulated, the aesthetic design of a graphical user interface (GUI) can be degraded due to the modulated texture of the visual stimulus interfering with the graphical content integrating the visual stimulus. For example, a blinking texture superimposed on a visual element of a GUI can occlude other background textures applied to the visual element. Accordingly, it may be advantageous to provide techniques for modulating visual stimuli for use with a BCI that preserve (or even improve) the neurologically detectable (e.g., by EEG) stimulation effects required by the BCI while also rendering the modulation less visible or less noticeable to a user.

Examples described herein use a binocular display (e.g., a pair of near-eye displays of an XR headset) to present a visual stimulus dichoptically. “Dichoptic” indicates a scenario in which each eye of a viewer views a separate and independent field. As used herein, a dichoptic visual stimulus refers to distinct visual stimuli presented to each of a user's two eyes, and wherein the two distinct visual stimuli differ from each other at least some of the time. In particular, examples described herein provide a dichoptic visual stimulus in which the visual stimulus provided to one eye is modulated (e.g., temporally modulated), whereas the visual stimulus provided to the other eye is not modulated.

In some examples, a first visual stimulus is presented to a first eye (e.g., to the left eye or right eye on a left or right near-eye display). The first visual stimulus is modulated according to existing BCI techniques: e.g., a texture or other visual component of the first visual stimulus is temporally modulated in accordance with a stimulus modulation scheme for the GUI. At the same time, a second visual stimulus is presented to a second eye (e.g., the other of the left eye or right eye, on the other of the left or right near-eye display). The second visual stimulus is not modulated in accordance with the stimulus modulation scheme for the GUI. In some cases, the second visual stimulus can be presented as a static, unmodulated stimulus; in other cases, the second visual stimulus can be presented with modulation applied that is different from the first modulation applied to the first visual stimulus. Various examples of modulation schemes for the first visual stimulus and second visual stimulus are described herein.

Experimental testing of BCI using a dichoptic visual stimulus has shown reduced visibility and awareness of the modulation of the first visual stimulus (relative to approaches in which both visual stimuli are modulated, or a single visual stimulus viewed by both eyes is modulated), while also preserving the accuracy of the BCI in detecting visual focus on a target object based on neural signal processing. The use of a dichoptic visual stimulus can, in some cases, create an illusion that fully or partially masks or hides the blinking or other modulation of the first visual stimulus, resulting in perception by the user of a static, unmodulated visual element when focusing the first eye and second eye on the first visual stimulus and second visual stimulus, respectively. The use of the dichoptic visual stimulus also results in a strong EEG response in the user's brain that can be processed to associate the EEG response with the characteristic modulation of the first visual stimulus. Approximately 70% of users tested using an AR display, a BCI, and dichoptic visual stimulus exhibited positive results across all metrics: reliable BCI performance due to a strong and maintained EEG response, without any reported discomfort or disturbance of visual perception caused by the blinking visual texture of the first visual stimulus. It will be appreciated that some examples described herein may be capable of producing wholly or mostly positive results for more than 70% of users.

In some examples, the textures applied to the first visual stimulus and second visual stimulus overlap perfectly when users focus on the visual element to which the textures are applied (e.g., a GUI element corresponding to the first visual stimulus and second visual stimulus). As a result, the static texture (or differently-modulated texture) applied to the second visual stimulus can completely or almost completely mask the blinking texture presented to the first eye, resulting in the perception of a static texture.

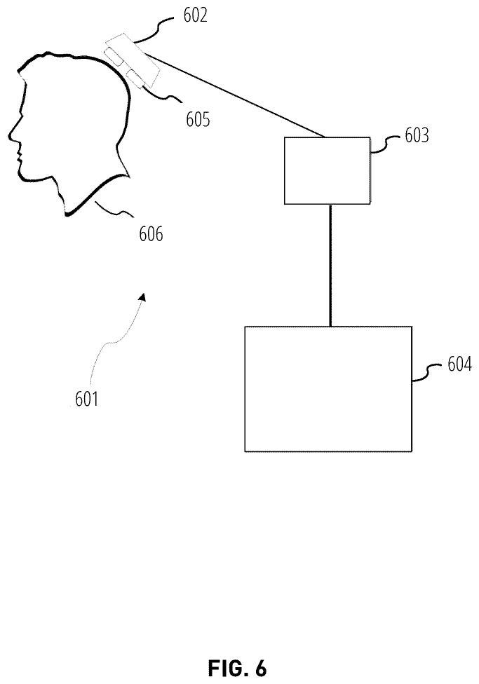

Examples of BCI techniques, including stimulus modulation schemes used for temporal modulation of visual stimuli, are described below with reference to FIG. 6 to FIG. 12. In some cases, the cyclic frequency with which a temporally modulated visual stimulus cycles through two or more states (e.g., a first state in which the texture is displayed and a second state in which the texture is not displayed) can be adjusted to various values based on user preferences or user calibration. In some examples, the cyclic frequency is set to values of 6 Hz or higher. In examples using a dichoptic visual stimulus, the choice of which of the user's eyes to designate as the first eye (viewing the modulated first visual stimulus) may be configurable based on device settings: for example, the user's dominant eye can be chosen as the first eye. A value for the cyclic frequency can then be selected, e.g., by device calibration using the user's first eye.

In some cases, some users may encounter challenges when using a BCI that uses a dichoptic visual stimulus for stimulus modulation. For example, some users (e.g., some subset of the 30% of users encountering imperfect results in experimental testing) may not generate a strong enough EEG response to the dichoptic visual stimulus to allow the BCI to reliably detect visual focus on the target object corresponding to the dichoptic visual stimulus. In some cases, these users may be able to improve their EEG response, and therefore BCI sensitivity and/or accuracy, by closing the second eye viewing the (unmodulated, or differently-modulated) second visual stimulus. Thus, in some cases, a user may be prompted or trained to wink the second eye (e.g., the non-dominant eye) to intentionally select a GUI element being viewed by the first eye.

The description that follows includes systems, methods, techniques, instruction sequences, and computing machine program products that embody illustrative embodiments of the disclosure. In the following description, for the purposes of explanation, numerous specific details are set forth in order to provide an understanding of various embodiments of the inventive subject matter. It will be evident, however, to those skilled in the art, that embodiments of the inventive subject matter may be practiced without these specific details. In general, well-known instruction instances, protocols, structures, and techniques are not necessarily shown in detail.

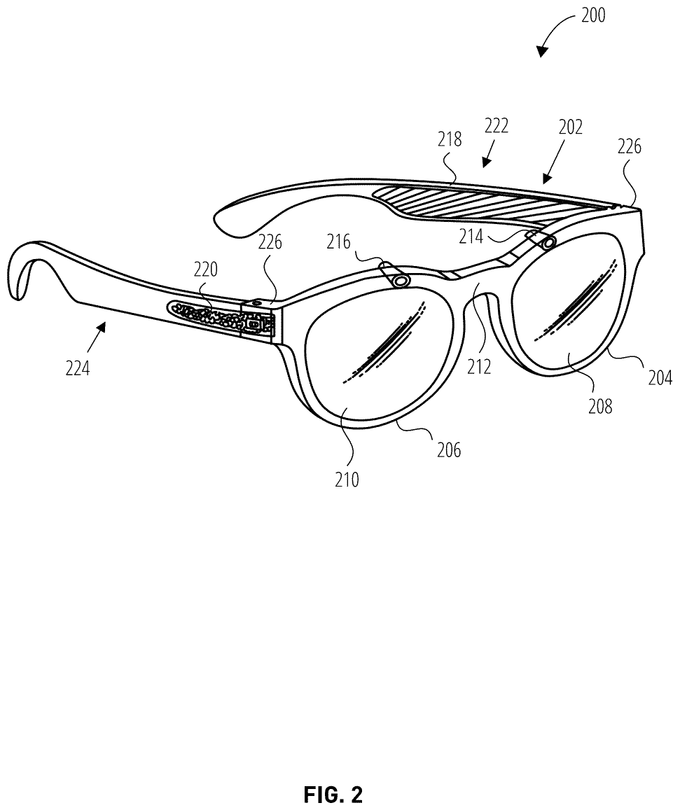

FIG. 2 is perspective view of a head-worn AR device (e.g., glasses 200), in accordance with some examples. The glasses 200 can include a frame 202 made from any suitable material such as plastic or metal, including any suitable shape memory alloy. In one or more examples, the frame 202 includes a first or left optical element holder 204 (e.g., a display or lens holder) and a second or right optical element holder 206 connected by a bridge 212. A first or left optical element 208 and a second or right optical element 210 can be provided within respective left optical element holder 204 and right optical element holder 206. The right optical element 210 and the left optical element 208 can be a lens, a display, a display assembly, or a combination of the foregoing. Any suitable display assembly can be provided in the glasses 200.

The frame 202 additionally includes a left arm or temple piece 222 and a right arm or temple piece 224. In some examples the frame 202 can be formed from a single piece of material so as to have a unitary or integral construction.

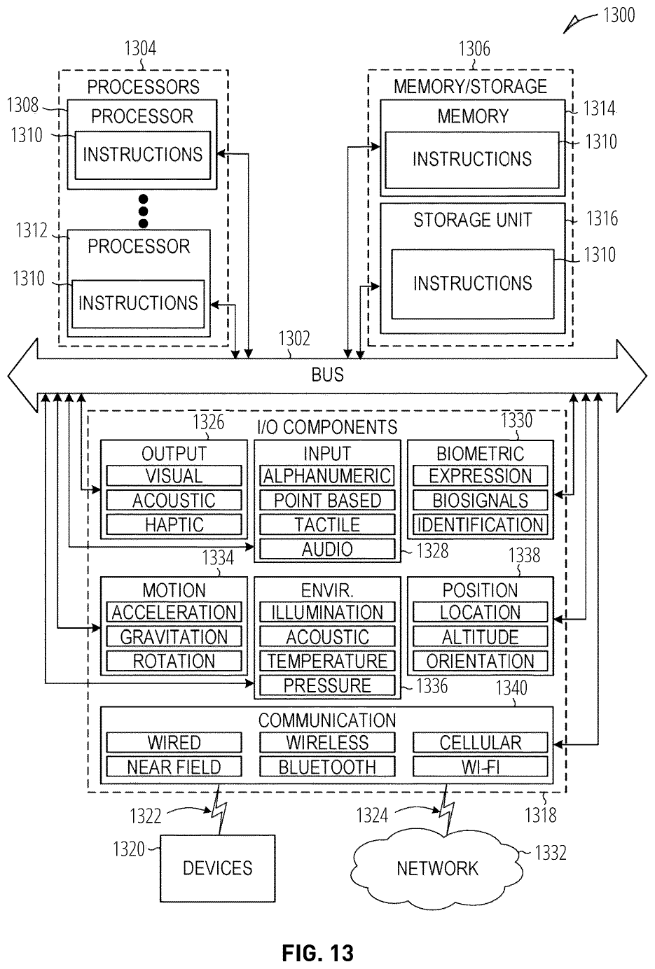

The glasses 200 can include a computing device, such as a computer 220, which can be of any suitable type so as to be carried by the frame 202 and, in one or more examples, of a suitable size and shape, so as to be partially disposed in one of the temple piece 222 or the temple piece 224. The computer 220 can include one or more processors with memory, wireless communication circuitry, and a power source. Various other examples may include these elements in different configurations or integrated together in different ways. In some examples, the computer 220 can implement some or all of the functions of a computing system configured to perform methods and operations described herein, such as the machine 1300 described in reference to FIG. 13 below.

The computer 220 additionally includes a battery 218 or other suitable portable power supply. In some examples, the battery 218 is disposed in left temple piece 222 and is electrically coupled to the computer 220 disposed in the right temple piece 224. The glasses 200 can include a connector or port (not shown) suitable for charging the battery 218, a wireless receiver, transmitter or transceiver (not shown), or a combination of such devices.

The glasses 200 include a first or left camera 214 and a second or right camera 216. Although two cameras are depicted, other examples contemplate the use of a single or additional (i.e., more than two) cameras. In one or more examples, the glasses 200 include any number of input sensors or other input/output devices in addition to the left camera 214 and the right camera 216, such as one or more optical calibration sensors, eye tracking sensors, ambient light sensors, and/or environment sensors, as described below. Such sensors or input/output devices can additionally include location sensors, motion sensors, and so forth. In some examples, the optical calibration sensors, eye tracking sensors, ambient light sensors, and/or environment sensors can include the left camera 214 and/or right camera 216: for example, the left camera 214 and/or right camera 216 may be used as the ambient light sensor to detect ambient light, and may also be used as at least part of a suite of environment sensors to detect environmental conditions around a user of the glasses 200. It will be appreciated that the cameras 214, 216 are a form of optical sensor, and that the glasses 200 may include additional types of optical sensors in some examples.

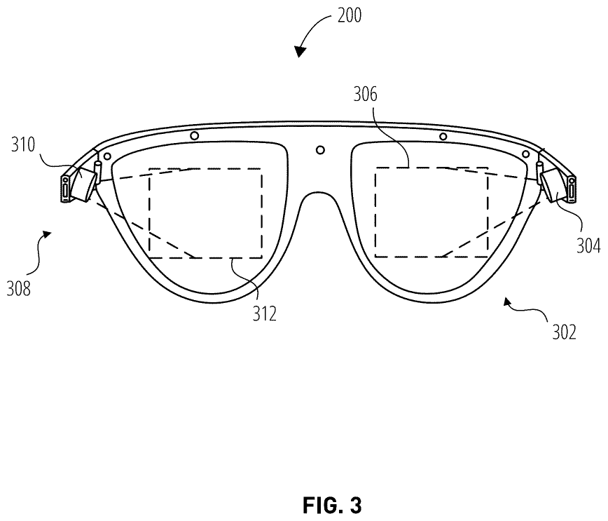

FIG. 3 illustrates the glasses 200 from the perspective of a user. For clarity, a number of the elements shown in FIG. 2 have been omitted. As described in FIG. 2, the glasses 200 shown in FIG. 3 include left optical element 208 and right optical element 210 secured within the left optical element holder 204 and the right optical element holder 206 respectively.

The glasses 200 include right forward optical assembly 302 comprising a right projector 304 and a right image presentation component 306, and a left forward optical assembly 308 including a left projector 310 and a left image presentation component 312. The right forward optical assembly 302 may also be referred to herein, by itself or in combination with the right optical element 210, as a right near-eye optical see-through XR display or simply a right near-eye display. The left forward optical assembly 308 may also be referred to herein, by itself or in combination with the left optical element 208, as a left near-eye optical see-through XR display or simply a left near-eye display.

In some examples, the image presentation components 306 are waveguides. The waveguides include reflective or diffractive structures (e.g., gratings and/or optical elements such as mirrors, lenses, or prisms). Projected light emitted by the projector 304 encounters the diffractive structures of the waveguide of the image presentation component 306, which directs the light towards the right eye of a user to provide an image on or in the right optical element 210 that overlays the view of the real world seen by the user. Similarly, projected light emitted by the projector 310 encounters the diffractive structures of the waveguide of the image presentation component 312, which directs the light towards the left eye of a user to provide an image on or in the left optical element 208 that overlays the view of the real world seen by the user. The combination of a GPU, the right forward optical assembly 302, the left optical element 208, and the right optical element 210 provide an optical engine of the glasses 200. The glasses 200 use the optical engine to generate an overlay of the real world view of the user including display of a 3D user interface to the user of the glasses 200. The surface of the optical element 208 or 210 from which the projected light exits toward the user's eye is referred to as a user-facing surface or an image presentation surface of the near-eye optical see-through XR display.

It will be appreciated that other display technologies or configurations may be utilized within an optical engine to display an image to a user in the user's field of view. For example, instead of a projector 304 and a waveguide, an LCD, LED or other display panel or surface may be provided.

In use, a user of the glasses 200 will be presented with information, content and various 3D user interfaces on the near eye displays. As described in more detail herein, the user can then interact with the glasses 200 using the buttons 226, voice inputs or touch inputs on an associated device, and/or hand movements, locations, and positions detected by the glasses 200. In some examples, as described below, a user may also provide control input to the glasses 200 using a BCI.

In some examples, one or more further optical lenses may be used to adjust the presentation of the virtual content to the user's eye. For example, lenses can be placed on the user-facing side and/or the exterior side of the image presentation component (e.g., image presentation component 306 or 312) to modulate the plane in front of the user's eye where that the virtual content appears, i.e., to adjust the perceived distance of the virtual content from the user's eye by adjusting the focal distance of the virtual content. The near user-facing side lens affects the perceived distance of the virtual content in front of the user; while the exterior side lens is provided to neutralize the effect of the near side lens on real-world objects. In some examples, an ophthalmic lens can be positioned on the user-facing side of the image presentation component (e.g., image presentation component 306 or 312) to allow users needing visual correction to correctly perceive the virtual content. In some examples, dynamic ophthalmic lenses can be used that are configured to vary the focal distance of the displayed virtual content.

It will be appreciated that examples described herein can be combined with various XR display designs.

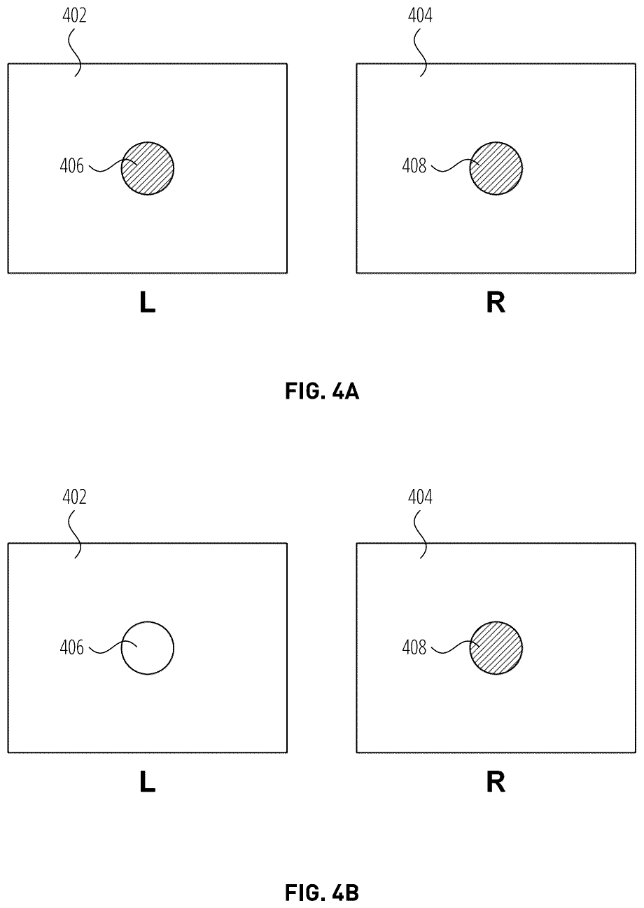

FIG. 4A is a rear view (e.g., the same view as FIG. 3) of the eye-facing sides of a left near-eye display 402 and a right near-eye display 404 presenting a dichoptic visual stimulus. The dichoptic visual stimulus includes a first visual stimulus 406 presented on the left near-eye display 402 and a second visual stimulus 408 presented on the right near-eye display 404. Jointly, the first visual stimulus 406 and second visual stimulus 408 correspond to a single visual element when viewed by both of the user's eyes; as used herein, a visual element may refer to anything visually perceived by a user, and may include GUI elements, virtual objects, or other visual elements referred to herein. When incorporating a modulated visual stimulus for use with a BCI, a visual element may be referred to as a target object in the context of using a BCI to detect visual focus on the visual element.

In the example view of FIG. 4A, the left near-eye display 402 presents the first visual stimulus 406 of the dichoptic visual stimulus in a first state. The first state may be, for example, a state in which the first visual stimulus 406 is presented with a stimulation texture applied to it, or otherwise including a distinct visual component.

The right near-eye display 404 presents the second visual stimulus 408. The second visual stimulus 408 is either unmodulated, or modulated according to a different modulation from the first modulation applied to the first visual stimulus 406. In this example, the second visual stimulus 408 is displayed with the stimulation texture applied to it, to match the presentation of the first visual stimulus 406 in its first state. However, it will be appreciated that, in some examples, the second visual stimulus 408 is always presented without a stimulation texture applied to it, such that the second visual stimulus 408 matches the first visual stimulus 406 as presented in its second state (described below with reference to FIG. 4B).

The point of view of FIG. 4A is roughly that of the user's eyes; the first visual stimulus 406 presented by the left near-eye display 402 is presented to the user's left eye, and the second visual stimulus 408 presented by the right near-eye display 404 is presented to the user's right eye.

It will be appreciated that the left near-eye display 402 and right near-eye display 404 may be implemented as screen-based displays (e.g., LCD or LED screens), as waveguide-based displays, or as any other suitable types of displays. In some examples, the visual characteristics of the first visual stimulus 406 and second visual stimulus 408 (e.g., pixel coordinates of different portions of the displayed visual element) may be determined based on the operating principles of a binocular display, such as an XR display, to cause the user to perceive the visual element corresponding to the first visual stimulus 406 and second visual stimulus 408 at a given depth in 3D space and/or having various 3D characteristics. This may result in further discrepancies between the presentation of the first visual stimulus 406 and second visual stimulus 408 of the dichoptic visual stimulus that are unrelated to the modulation differences used for BCI that are described herein.

FIG. 4B shows the left near-eye display 402 of FIG. 4A presenting the first visual stimulus 406 in a second state, and the right near-eye display 404 of FIG. 4A presenting the second visual stimulus 408 in the same static state shown in FIG. 4B. In this example, the second state of the first visual stimulus 406 is one in which the stimulation texture is not applied to the first visual stimulus 406, such that the first visual stimulus 406 is displayed without the texture or other visual component that is displayed in the first state.

It will be appreciated that different modulation schemes may be used in different examples, other than the temporally cyclic or alternating presentation of two states of the first visual stimulus 406. In some examples, more than two states may be used in temporally modulating the first visual stimulus 406, and the modulation scheme may cycle among these three or more states by displaying each according to a respective duty cycle or time period within the modulation cycle. In some examples, the two or more states of the first visual stimulus 406 are distinguished from each other by some visual characteristic other than the presence or absence of a texture or other visual component: for example, visual or optical characteristics such as the light spectrum distribution, brightness, depth, resolution, or any other visual characteristic could potentially be varied or modulated in a way that this variation or modulation would be detectable through EG signal processing. The examples described herein are not intended to be limited to a particular temporal modulation scheme for modulation of the first visual stimulus 406 of one or more target objects used by a BCI.

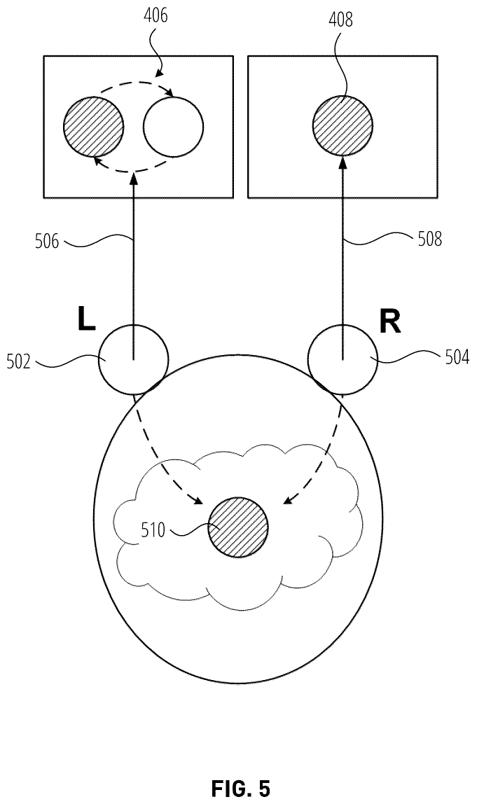

FIG. 5 is a schematic representation of the dichoptic visual stimulus of FIG. 4A and FIG. 4B, as presented to left eye 502 and right eye 504 of a user. The first visual stimulus 406 alternates or cycles between the first state and second state at different times in its modulation cycle. The second visual stimulus 408 is static in this example.

When the user's left gaze vector 506 focuses on the first visual stimulus 406 and the user's right gaze vector 508 focuses on the second visual stimulus 408, the user perceives the first visual stimulus 406 and second visual stimulus 408 as corresponding to a single visual element. Specifically, in some cases (as described above), the user will be conscious of a visual percept 510 of the single visual element that corresponds to the static, unmodulated state of the second visual stimulus 408. In some cases, two different modulations applied to the first visual stimulus 406 and second visual stimulus 408 interfere with and mask each other, such that the user will be conscious of a visual percept 510 of a static version of the single visual element. In some cases, visual characteristics of the first visual stimulus 406 other than its modulation may shape the visual percept 510, such as differences in point of view of a 3D object that contribute to depth perception when combined with the visual presentation of second visual stimulus 408, while still masking the modulation of the first visual stimulus 406 to the user's conscious perception. Thus, in some examples, the visual percept 510 formed by the user's brain in response to the dichoptic visual stimulus corresponds to a static object undisturbed by the modulation of first visual stimulus 406, while unconscious portions of the visual processing apparatus of the user's brain react to the modulation of the first visual stimulus 406 with sufficient strength for this reaction to be detectable by a BCI through EEG signal collection and processing.

FIG. 6 illustrates an example of an electronic architecture for the reception and processing of EEG signals by means of an EEG device 601 according to the present disclosure.

In some examples, the EEG device 601 may be referred to herein as a neural signal capture device. However, it will be appreciated that some examples described herein could use a neural signal capture device of a different type or using different neural signal capture methodologies.

To measure diffuse electric potentials on the surface of the skull of a subject 606, the EEG device 601 includes a portable device 602 (i.e. a cap or headpiece), analog-digital conversion (ADC) circuit 603 and a microcontroller 604. The portable device 602 of FIG. 6 includes one or more electrodes 605, typically between 1 and 128 electrodes, such as between 2 and 64, such as between 4 and 16.

Each electrode 605 may comprise a sensor for detecting the electrical signals generated by the neuronal activity of the subject and an electronic circuit for pre-processing (e.g. filtering and/or amplifying) the detected signal before analog-digital conversion: such electrodes being termed “active”. The electrodes 605 are shown in use in FIG. 6, where the sensor is in physical proximity with the subject's scalp. The electrodes may be suitable for use with a conductive gel or other conductive liquid (termed “wet” electrodes) or without such liquids (termed “dry” electrodes).

Each ADC circuit 603 is configured to convert the signals of a given number of electrodes 605, for example between 1 and 128.

The ADC circuits 603 are controlled by the microcontroller 604 and communicate with it, for example, by the SPI (“Serial Peripheral Interface”) protocol. The microcontroller 604 packages the received data for transmission to an external processing unit (not shown), for example a computer (such as computer 220 of the glasses 200), a mobile phone, a virtual reality headset, an automotive or aeronautical computer system, for example a car computer or a computing system, by a wired or wireless communication link, for example by Bluetooth, Wi-Fi (“Wireless Fidelity”) or Li-Fi (“Light Fidelity”).

In certain embodiments, each active electrode 605 is powered by a battery (not shown in FIG. 6). The battery can be provided in a housing of the portable device 602.

In certain embodiments, each active electrode 605 measures a respective electric potential value from which the potential measured by a reference electrode (Ei=Vi−Vref) is subtracted, and this difference value is digitized by means of the ADC circuit 603 then transmitted by the microcontroller 604.

In certain embodiments, the methods described herein introduce target objects (e.g., visual stimuli and/or their visible features) for display in a graphical user interface of a display device. The target objects include control items and the control items are in turn associated with user-selectable actions.

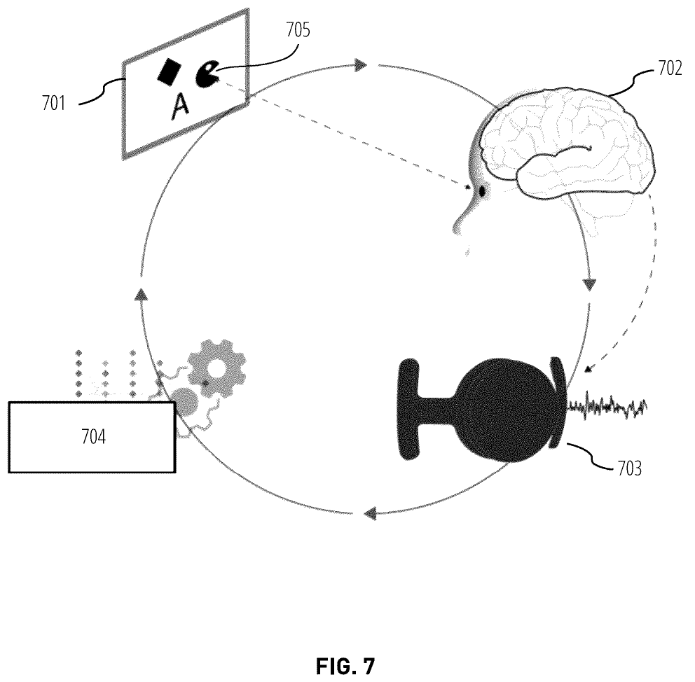

FIG. 7 illustrates a computing system incorporating a brain computer interface (BCI) according to the present disclosure. The computing system incorporates a neural signal capture device 703, such as the EEG device 601 illustrated in FIG. 6. In the computing system, an image is displayed on at least one display of a display device 701, such as the left near-eye displays and right near-eye display of the glasses 200 of FIG. 2. In some examples, the computing system includes a display device such as the glasses 200, the BCI shown in FIG. 7, and optionally one or more computing devices in addition to the computer 220 of the glasses 200. The subject 702 (also referred to as a user) views the image on at least one display of a display device 701 (such as a binocular display device including the left near-eye display 402 and right near-eye display 404), focusing on a target object 705.

In some examples, the display device 701 displays at least the target object 705 (e.g., a dichoptic visual stimulus including the first visual stimulus 406 and second visual stimulus 408 of FIG. 4A and FIG. 4B) as a graphical object with a varying temporal characteristic distinct from the temporal characteristic of other displayed objects and/or the background in the display. The varying temporal characteristic may be, for example, a constant or time-locked flickering effect altering the appearance of the target object at a rate greater than 6 Hz. In some examples, more than one graphical object is a potential target object: e.g., the viewing subject is offered a choice of target object to focus attention on, such as two different instances of dichoptic visual stimulus, each having a first visual stimulus 406 modulated according to a distinct modulation and a second visual stimulus 408. In such examples, each target object is associated with a distinct spatial and/or temporal code.

The neural signal capture device 703 detects neural responses (i.e. tiny electrical potentials indicative of brain activity in the visual cortex) associated with attention focused on the target object; the visual perception of the varying temporal characteristic of the target object(s) therefore acts as a stimulus in the subject's brain, generating a specific brain response that accords with the code associated with the target object in attention. The detected neural responses (e.g. electrical potentials) are then converted into signals and transferred to a processing device 704 for decoding. Examples of neural responses include visual evoked potentials (VEPs), which are commonly used in neuroscience research. The term VEPs encompasses conventional SSVEPs, as mentioned above, where stimuli oscillate at a specific frequency and other methods such as the code-modulated VEP, stimuli are subject to a variable or pseudo-random temporal code.

The processing device 704 executes instructions that interpret the received neural signals to determine feedback indicating the target object having the current focus of (visual) attention (e.g., both eyes having their gaze converged on the target object, and both eyes being focused on the target object) in real time. Decoding the information in the neural response signals relies upon a correspondence between that information and one or more aspects of the temporal profile of the target object (i.e. the stimulus).

In certain embodiments, the processing device may conveniently generate the image data presented on the display device 701 including the temporally varying target object.

The feedback may conveniently be presented visually on the display screen. For example, the display device may display an icon, cursor, crosshair or other graphical object or effect in close proximity to the target object (or overlapping or at least partially occluding that object), highlighting the object that appears to be the current focus of visual attention. Clearly, the visual display of such feedback has a reflexive cognitive effect on the perception of the target object, amplifying the brain response. This positive feedback (where the apparent target object is confirmed as the intended target object by virtue of prolonged amplified attention) is referred to herein as “neurosynchrony”.

FIG. 8 illustrates the use of a neural response device such as that in FIG. 6 and FIG. 7 in discriminating between a plurality of target objects. The neural response device worn by the user (e.g. user 802 in FIG. 8) is an electrode helmet for an EEG device (such as EEG device 601). Here, the user wearing the helmet views a screen 801 displaying a plurality of target objects (the digits in an on-screen keypad), which are blinking at distinctly different times, frequencies and/or duty cycles. The electrode helmet can convey a signal derived from the user's neural activity. Here, the user is focusing on the digit “5”, 803, where at time t1 the digit “3”, 805, blinks, at time t2 the digit “4”, 806, blinks, at time t3 the digit “5”, 804, blinks, and at time t4, the digit “6”, 807, blinks. The neural activity as conveyed by the helmet signal would be distinctly different at t3 than at the other points in time. That is because the user is focusing on digit “5”, 803, which blinks on, 804, at t3. However, to differentiate that signal occurring at t3 with those at the other times, all the objects on the screen must blink at distinctively different times. Thus, the screen would be alive with blinking objects making for an uncomfortable viewing experience.

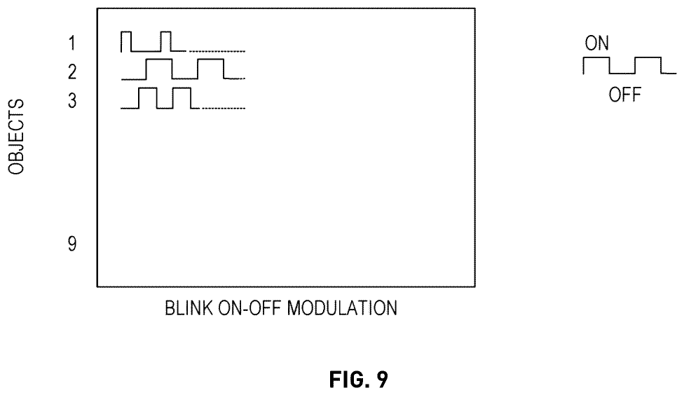

The system in FIG. 8 could be using a display signal pattern such as the exemplary pattern shown in FIG. 9 where the screen objects will blink at different points in time, with different cyclic frequencies and/or duty cycles.

It will be appreciated that, whereas the BCI techniques described herein use a temporal on/off blinking modulation scheme to associate distinct and characteristic temporal modulation to each visual stimulus (e.g., each digit of the on-screen keyboard of FIG. 8), in some examples other visual modulation schemes can be used to enable a BCI device to decode neural signals to detect when visual attention is focused on a given visual stimulus. For example, different temporal modulation waveforms from the pattern shown in FIG. 9 can be used in some cases, such as sinusoidal wave patterns having various frequencies and phases, minimally correlated signals, and so on.

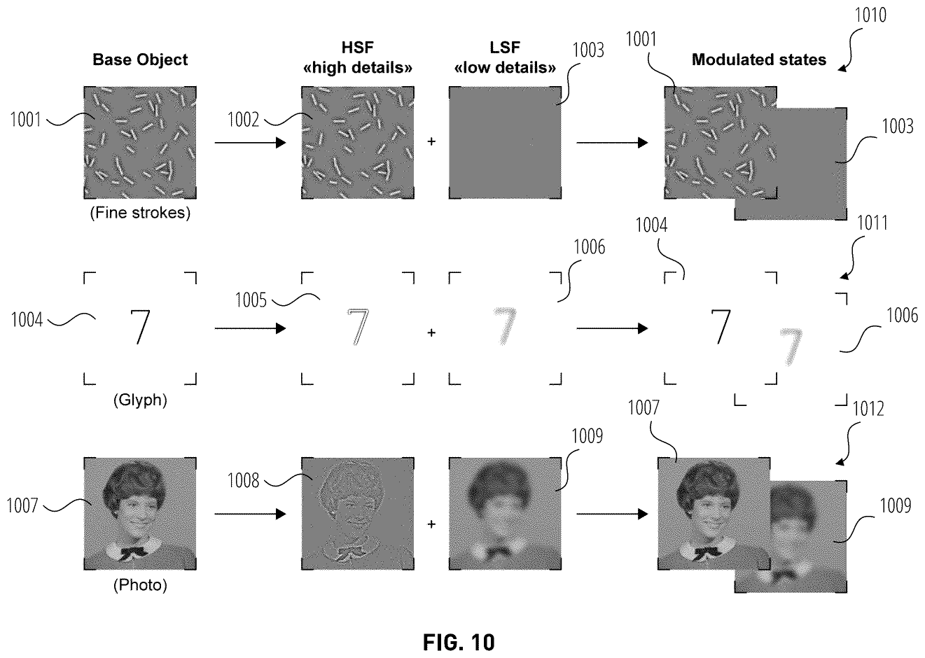

FIG. 10 shows three different screen display objects: a cluster of multiple fine strokes 1001, each with respective, different orientations; a glyph 1004 (shown as the digit “7”); and a photograph 1007. These screen display objects (also referred to herein as screen objects, target objects, or visual elements) illustrate example temporal modulation schemes in which the spatial frequency of visual features or components of the screen objects is used in generating the modulated visual stimuli.

In some examples, image data for screen display objects is processed to extract a version of each screen display object composed of only high spatial frequency (HSF) components for each object. One or more screen display objects within the field of view of a user can be of potential interest for use in a BCI-based GUI (e.g., target objects). The screen display objects can be processed by extracting components that relate to visual properties of the objects (e.g., their edges), and applying a modulation to the high spatial frequency component of those visual properties. Thus, a blinking visual stimulus used to elicit neural responses, such as visual evoked potentials (VEPs), can be conveyed only through the HSF version of the objects. The modulation makes the object blink or otherwise visually alter so that the modulation acts as a stimulus for a correlated neural response. The neural response may in turn be measured and decoded to determine which object of interest is the focus of the user's attention.

In some examples, the image data may further be processed to extract a further version of the object composed only of the low spatial frequency (LSF) components. In some examples, where an LSF version is extracted, the modulated HSF version may be superimposed on the LSF version (which does not blink).

By filtering these objects, one may produce an HSF version of each (denoted 1002, 1005, and 1008 respectively) and an LSF version of each (denoted 1003, 1006, and 1009 respectively). The human vision system is naturally more responsive to HSF signals from objects of focus (e.g., objects close to the center of the field of vision or within the foveal region), and to LSF signals from peripheral objects (e.g., objects close to the periphery of the field of vision or outside the foveal region). As a result, if a display is dissociated into HSF and LSF versions, and only the HSF versions are made to blink whereas the LSF versions are constantly displayed, the peripheral object “noise” produced by LSF can be subdued by keeping the LSF version of each object constant, and only blinking the HSF versions of each object. This effectively reduces LSF noise from peripheral objects and reduces LSF signal from an object under focus. Since the human vision system naturally associates HSF with foveal objects (those under focus), keeping the LSF version constant will have essentially no effect on the neural signals stimulated by the object under focus but will have reduced LSF interference from objects on the periphery. It has been observed that, when the screen display objects are multiple fine strokes (as in fine strokes 1001), the HSF version 1002 of the base object (fine strokes 1001) effectively provides a particularly high possible spatial frequency (while remaining fully visible) relative to the base object, whereas the LSF version 1003 of fine strokes 1001 offers a particularly low possible spatial frequency (as it is a plain uniform background).

Thus, a visual stimulus can be generated wherein two states of a screen display object are shown in an alternating cycle or otherwise time-modulated. A modulated version 1010 of the fine strokes 1001 (cycling between the base object of the fine strokes 1001 and the LSF version 1003 thereof), a modulated version 1011 of glyph 1004 (cycling between the base object of the glyph 1004 and the LSF version 1006 thereof), and a modulated version 1012 of photograph 1007 (cycling between the base object of the photograph 1007 and the LSF version 1009 thereof) are shown, any of which could be used as a modulated first visual stimulus 406 in examples described herein. Because only one version is blinking (e.g., the HSF version), the apparent blinking of the object is more subdued for the peripheral objects whereas the blinking of the object of focus is apparent because the human vision system is more sensitive to HSF-evoking stimuli from objects of focus.

Thus, in the context of a dichoptic visual stimulus, the modulation applied to the first visual stimulus 406 (referred to herein as a “first modulation”) can be a type of temporal modulation whereby the first visual stimulus 406 is presented in at least two different states at each of at least two different times.

In some examples, the second visual stimulus 408 of a dichoptic visual stimulus corresponds to a base object version of the underlying display object, such as the fine strokes 1001, glyph 1004, or photograph 1007. The first visual stimulus 406, however, is temporally modulated such that it alternates between a first state in which it is presented as the base object (fine strokes 1001, glyph 1004, or photograph 1007), and a second state in which it is presented as the LSF version thereof (LSF version 1003, LSF version 1006, or LSF version 1009 respectively). Thus, the first visual stimulus 406 could be generated as modulated version 1012, which cycles between a first state presenting a first view of the visual element (e.g., the base object version of photograph 1007 as seen from the point of view of the first eye) and a second state in which the view of the visual element excludes one or more high spatial frequency visual features of the visual element (e.g., LSF version 1009, excluding the features of HSF version 1008). The second visual stimulus 408 is a static second view of the visual element (e.g., the base object version of photograph 1007 as seen from the point of view of the second eye).

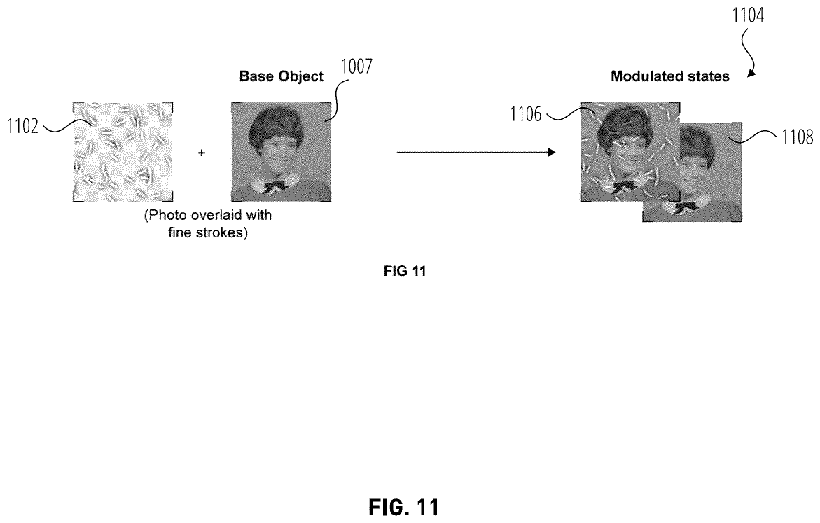

FIG. 11 illustrates a second example modulation scheme using a screen display object (shown as photograph 1007) and a texture (shown as texture 1102) to generate a modulated version 1104 combining the screen display object and the texture 1102. In the illustrated example, the modulated version 1104 has a first state 1108 showing the screen display object, and a second state 1106 showing the screen display object overlaid with, or otherwise combined with, the texture 1102. Thus, whereas the examples shown in FIG. 10 have modulated stimuli in which a base object has HSF features included or omitted in two temporally cycled states, the example shown in FIG. 11 has a modulated stimulus in which a base object has a visual texture or pattern overlaid or combined with it, or not, in two temporally cycled states.

In some examples, therefore, a temporally modulated pattern, texture, or other visual component is applied to the underlying visual element of the first visual stimulus 406, and a static version of the pattern, texture, or other visual component is applied to the underlying visual element of the second visual stimulus 408. Thus, for example, a texture 1102 or pattern can be applied to (e.g., overlaid or otherwise combined with) a base visual element of the first visual stimulus 406 in temporal modulation, cycling between two states of the modulated version 1104. For example, the modulated version 1104 could cycle between a first state 1108, in which no texture is overlaid on the visual element, and a second state 1106, in which the texture 1102 is overlaid on the visual element. In other words, the first visual stimulus 406 is cycled between a first state 1108 presenting a first view of the visual element, and a second state 1106 in which the view of the visual element is overlaid with a second visual texture or pattern (e.g., texture 1102). The second visual stimulus 408 is a static second view of the visual element, overlaid with a static pattern or texture (e.g., texture 1102).

Alternatively, in other examples, the modulated version 1104 used for the first visual stimulus 406 can cycle between a first state in which a first texture is overlaid on the visual element, and a second state in which a second texture is overlaid on the visual element. The second visual stimulus 408 used in such an example can be a static second view of the visual element, overlaid with a static version of the first texture.

In some examples, both the first visual stimulus 406 and second visual stimulus 408 are modulated, but each is modulated according to a different modulation scheme, such that the dichoptic modulation masks the visibility of the flickering or other modulation effect to the user's perception. For example, the first visual stimulus 406 can be modulated by blinking a pattern on and off according to a given frequency and phase; concurrently, the second visual stimulus 408 can be modulated by blinking the same pattern according to the same frequency but an inverted phase, or a visually inverted version of the pattern (e.g., color inverted, or LSF instead of HSF) at the same frequency and phase. In some examples, the first visual stimulus 406 and second visual stimulus 408 are modulated using the same pattern applied at the same frequency and phase, but the visual characteristics of the pattern are applied with less visibility to the second visual stimulus 408 (e.g., with a lower contrast or luminance) than they are to the first visual stimulus 406.

In some examples, the first visual stimulus 406 is modulated by varying the visibility of a visual component (e.g., the HSF component) over time, e.g., alternating between the LSF component being displayed at full contrast and/or luminance during a first time or duty cycle and the LSF component being displayed at partial contrast and/or luminance during a second time or duty cycle.

In some examples, the temporal modulation applied to the first visual stimulus 406 is a cyclic sequential presentation of at least two different states of the first visual stimulus 406 (e.g., the first state and second state) at a cyclic frequency of at least 6 Hz, as described above.

In certain embodiments, the modulation effects may be random or pseudo-random in nature: alterations in a visual characteristic of the object, such as blinking, can be performed at random times, e.g. following a pseudo-random temporal modulation pattern. Pseudo-random temporal patterns can be used, rather than strictly random patterns, to reduce temporal overlap between distinct temporal patterns associated with respective different objects.

In some examples, target objects (e.g., “screen objects” or “screen display objects”) are distinct graphical visual elements presented on a display of a display device, and overlay objects are generated to correspond to one or more of the screen objects. In some such examples, it is the overlay objects, rather than the screen objects themselves, which are filtered to produce HSF and LSF versions, and which are then modulated according to one of the schemes described herein.

Some of the modulation schemes described herein, such as temporal modulation schemes cycling HSF and/or LSF visual features on and off, or schemes modulating the application of textures, can be used in the context of a conventional (e.g., non-binocular) display to modulate a single visual stimulus seen by both of a user's eyes. However, as described above, modulation of a single visual stimulus seen by both eyes tends to result in degradation of visual perception when it is performed in such a way as to reliably trigger an EEG response detectable by a BCI. By using a binocular display to present a dichoptic stimulus in which only one eye's stimulus is modulated, or in which the two eyes' stimuli are modulated according to different schemes (e.g., different or inverse cyclic frequencies or duty cycles), examples described herein can effectively mask the modulation used for BCI, resulting in the user perceiving an unmodulated target object and thereby reducing the visibility of a visual modulation applied to the GUI elements or other visual stimuli used by the BCI to detect a user's visual attention or gaze.

If BCI visual stimulus visibility can be reduced or eliminated without causing user discomfort and while maintaining high levels of BCI reliability, it becomes feasible to use BCI in a wider range of use cases than is currently common. Ongoing, passive monitoring of a user's visual attention becomes more feasible when a user is not being presented with distracting, visibly blinking visual elements on a display. The reduced visibility and reduced salience of the BCI user interface thereby enables many practical applications that are currently limited by user comfort, usability, and human factors concerns.

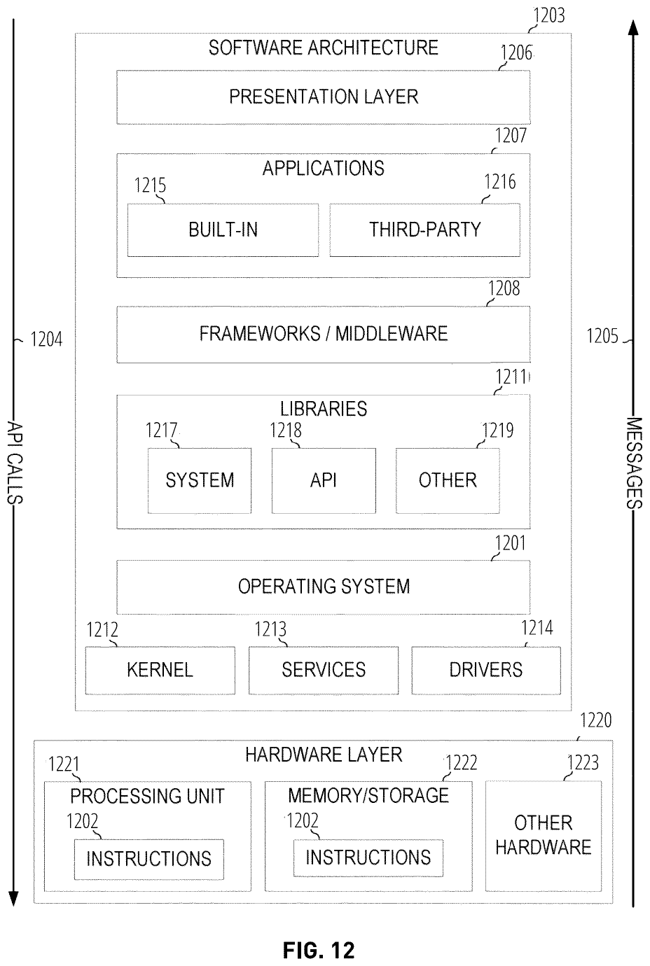

FIG. 12 is a block diagram illustrating an example software architecture 1203, which may be used in conjunction with various hardware architectures herein described. FIG. 12 is a non-limiting example of a software architecture and it will be appreciated that many other architectures may be implemented to facilitate the functionality described herein. The soft ware architecture 1203 may execute on hardware such as machine 1300 of FIG. 13 that includes, among other things, processors 1304, memory 1306, and input/output (I/O) components 1318. A representative hardware layer 1220 is illustrated and can represent, for example, the machine 1300 of FIG. 13. The representative hardware layer 1220 includes a processing unit 1221 having associated executable instructions 1202. The executable instructions 1202 represent the executable instructions of the software architecture 1203, including implementation of the methods, modules and so forth described herein. The hardware layer 1220 also includes memory and/or storage modules shown as memory/storage 1222, which also have the executable instructions 1202. The hardware layer 1220 may also comprise other hardware 1223, for example dedicated hardware for interfacing with EEG electrodes and/or for interfacing with display devices.

In the example architecture of FIG. 12, the software architecture 1203 may be conceptualized as a stack of layers where each layer provides particular functionality. For example, the software architecture 1203 may include layers such as an operating system 1201, libraries 1211, frameworks or middleware 1209, applications 1207 and a presentation layer 1206. Operationally, the applications 1207 and/or other components within the layers may invoke application programming interface (API) calls 1204 through the software stack and receive a response as messages 1205. The layers illustrated are representative in nature and not all software architectures have all layers. For example, some mobile or special purpose operating systems may not provide the frameworks/middleware 1208, while others may provide such a layer. Other software architectures may include additional or different layers.

The operating system 1201 may manage hardware resources and provide common services. The operating system 1201 may include, for example, a kernel 1212, services 1213, and drivers 1214. The kernel 1212 may act as an abstraction layer between the hardware and the other software layers. For example, the kernel 1212 may be responsible for memory management, processor management (e.g., scheduling), component management, networking, security settings, and so on. The services 1213 may provide other common services for the other software layers. The drivers 1214 may be responsible for controlling or interfacing with the underlying hardware. For instance, the drivers 1214 may include display drivers, EEG device drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 1211 may provide a common infrastructure that may be used by the applications 1207 and/or other components and/or layers. The libraries 1211 typically provide functionality that allows other software modules to perform tasks in an easier fashion than by interfacing directly with the underlying operating system 1201 functionality (e.g., kernel 1212, services 1213, and/or drivers 1214). The libraries 1211 may include system libraries 1217 (e.g., C standard library) that may provide functions such as memory allocation functions, string manipulation functions, mathematic functions, and the like. In addition, the libraries 1211 may include API libraries 1218 such as media libraries (e.g., libraries to support presentation and manipulation of various media formats such as MPEG4, H.264, MP3, AAC, AMR, JPG, and PNG), graphics libraries (e.g., an OpenGL framework that may be used to render 2D and 3D graphic content on a display), database libraries (e.g., SQLite that may provide various relational database functions), web libraries (e.g., WebKit that may provide web browsing functionality), and the like. The libraries 1211 may also include a wide variety of other libraries 1219 to provide many other APIs to the applications 1207 and other software components/modules.

The frameworks 1210 (also sometimes referred to as middleware) provide a higher-level common infrastructure that may be used by the applications 1207 and/or other software components/modules. For example, the frameworks/middleware 1208 may provide various graphic user interface (GUI) functions, high-level resource management, high-level location services, and so forth. The frameworks/middleware 1208 may provide a broad spectrum of other APIs that may be used by the applications 1207 and/or other software components/modules, some of which may be specific to a particular operating system or platform.

The applications 1207 include built-in applications 1215 and/or third-party applications 1216.

The applications 1207 may use built-in operating system functions (e.g., kernel 1212, services 1213, and/or drivers 1214), libraries 1211, or frameworks/middleware 1208 to create user interfaces to interact with users of the system. Alternatively, or additionally, in some systems interactions with a user may occur through a presentation layer, such as the presentation layer 1206. In these systems, the application/module “logic” can be separated from the aspects of the application/module that interact with a user.

FIG. 13 is a block diagram illustrating components of a machine 1300, according to some example embodiments, able to read instructions from a machine-readable medium (e.g., a machine-readable storage medium such as a non-transitory computer-readable storage medium) and perform any one or more of the methodologies discussed herein. Specifically, FIG. 13 shows a diagrammatic representation of the machine 1300 in the example form of a computing system, within which instructions 1310 (e.g., software, a program, an application, an applet, an app, or other executable code) for causing the machine 1300 to perform any one or more of the methodologies discussed herein may be executed. As such, the instructions 1310 may be used to implement modules or components described herein. The instructions 1310 transform the general, non-programmed machine 1300 into a particular machine programmed to carry out the described and illustrated functions in the manner described. In alternative embodiments, the machine 1300 operates as a standalone device or may be coupled (e.g., networked) to other machines. In a networked deployment, the machine 1300 may operate in the capacity of a server machine or a client machine in a server-client network environment, or as a peer machine in a peer-to-peer (or distributed) network environment. The machine 1300 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1310, sequentially or otherwise, that specify actions to be taken by the machine 1300. Further, while only a single machine 1300 is illustrated, the term “machine” shall also be taken to include a collection of machines that individually or jointly execute the instructions 1310 to perform any one or more of the methodologies discussed herein.

The machine 1300 may include processors 1304, memory 1306, and input/output (I/O) components 1318, which may be configured to communicate with each other such as via a bus 1302. In an example embodiment, the processors 1304 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an Application Specific Integrated Circuit (ASIC), a Radio-Frequency Integrated Circuit (RFIC), another processor, or any suitable combination thereof) may include, for example, a processor 1308 and a processor 1312 that may execute the instructions 1310. The term “processor” is intended to include multi-core processor that may comprise two or more independent processors (sometimes referred to as “cores”) that may execute instructions contemporaneously. Although FIG. 13 shows multiple processors, the machine 1300 may include a single processor with a single core, a single processor with multiple cores (e.g., a multi-core processor), multiple processors with a single core, multiple processors with multiples cores, or any combination thereof.

The memory 1306 may include a memory 1314, such as a main memory, a static memory, or other memory storage, and a storage unit 1316, both accessible to the processors 1304 such as via the bus 1302. The storage unit 1316 and memory 1314 store the instructions 1310 embodying any one or more of the methodologies or functions described herein. The instructions 1310 may also reside, completely or partially, within the memory 1314, within the storage unit 1316, within at least one of the processors 1304 (e.g., within the processor's cache memory), or any suitable combination thereof, during execution thereof by the machine 1300. Accordingly, the memory 1314, the storage unit 1316, and the memory of processors 1304 are examples of machine-readable media.

As used herein, “machine-readable medium” means a device able to store instructions and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)), and/or any suitable combination thereof. The term “machine-readable medium” should be taken to include a single medium or multiple media (e.g., a centralized or distributed database, or associated caches and servers) able to store the instructions 1310. The term “machine-readable medium” shall also be taken to include any medium, or combination of multiple media, that is capable of storing instructions (e.g., instructions 1310) for execution by a machine (e.g., machine 1300), such that the instructions, when executed by one or more processors of the machine 1300 (e.g., processors 1304), cause the machine 1300 to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

The input/output (I/O) components 1318 may include a wide variety of components to receive input, provide output, produce output, transmit information, exchange information, capture measurements, and so on. The specific input/output (I/O) components 1318 that are included in a particular machine will depend on the type of machine. For example, user interface machines and portable machines such as mobile phones will likely include a touch input device or other such input mechanisms, while a headless server machine will likely not include such a touch input device. It will be appreciated that the input/output (I/O) components 1318 may include many other components that are not shown in FIG. 13.

The input/output (I/O) components 1318 are grouped according to functionality merely for simplifying the following discussion and the grouping is in no way limiting. In various example embodiments, the input/output (I/O) components 1318 may include output components 1326 and input components 1328. The output components 1326 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1328 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instruments), tactile input components (e.g., a physical button, a touch screen that provides location and/or force of touches or touch gestures, or other tactile input components), audio input components (e.g., a microphone), and the like.

In further example embodiments, the input/output (I/O) components 1318 may include biometric components 1330, motion components 1334, environment components 1336, or position components 1338 among a wide array of other components. For example, the biometric components 1330 may include components to detect expressions (e.g., hand expressions, facial expressions, vocal expressions, body gestures, or eye tracking), measure biosignals (e.g., blood pressure, heart rate, body temperature, perspiration, or brain waves, such as the output from an EEG device), identify a person (e.g., voice identification, retinal identification, facial identification, fingerprint identification, or electroencephalogram based identification), and the like. The motion components 1334 may include acceleration sensor components (e.g., accelerometer), gravitation sensor components, rotation sensor components (e.g., gyroscope), and so forth. The environmental environment components 1336 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detect concentrations of hazardous gases for safety or to measure pollutants in the atmosphere), or other components that may provide indications, measurements, or signals corresponding to a surrounding physical environment. The position components 1338 may include location sensor components (e.g., a Global Position System (GPS) receiver component), altitude sensor components (e.g., altimeters or barometers that detect air pressure from which altitude may be derived), orientation sensor components (e.g., magnetometers), and the like.

Communication may be implemented using a wide variety of technologies. The input/output (I/O) components 1318 may include communication components 1340 operable to couple the machine 1300 to a network 1332 or devices 1320 via a coupling 1324 and a coupling 1322 respectively. For example, the communication components 1340 may include a network interface component or other suitable device to interface with the network 1332. In further examples, communication components 1340 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1320 may be another machine or any of a wide variety of peripheral devices (e.g., a peripheral device coupled via a Universal Serial Bus (USB)). Where an EEG device or display device is not integral with the machine 1300, the device 1120 may be an EEG device and/or a display device.

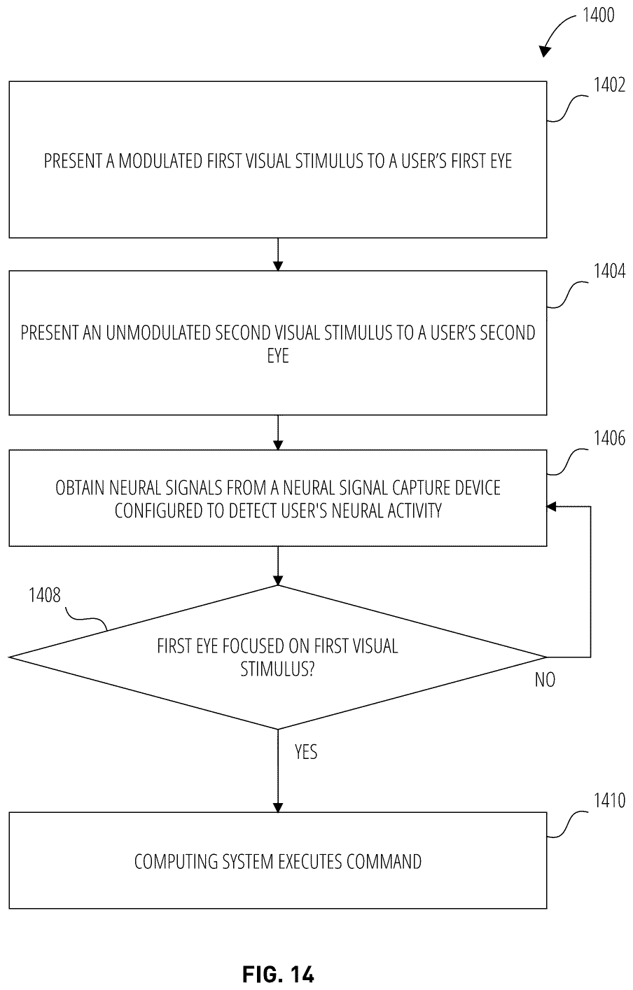

FIG. 14 is a flowchart illustrating operations of a method for method for visual BCI using dichoptic stimulation. Whereas the method 1400 is described in reference to the example BCIs, devices, systems, and visual stimuli illustrated in the foregoing figures, it will be appreciated that method 1400 can be performed by any suitable computing system having a binocular or stereoscopic display and a BCI.

Although the example method 1400 depicts a particular sequence of operations, the sequence may be altered without departing from the scope of the present disclosure. For example, some of the operations depicted may be performed in parallel or in a different sequence that does not materially affect the function of the method 1400. In other examples, different components of an example device or system that implements the method 1400 may perform functions at substantially the same time or in a specific sequence.

In some examples, the operations of method 1400 are performed continuously in a loop to provide a continuous indication of the focus of the user's visual attention during a human-computer interaction session. This indication of visual attention can be used as an input to a computing system to control the behavior of the computing system.

According to some examples, the method 1400 includes presenting a first visual stimulus 406 to a user's first eye (e.g., the left eye 502 or right eye 504) at operation 1402. As described above, the first visual stimulus 406 is presented with a first modulation, such as one of the various modulations described above with reference to FIG. 4A-FIG. 4B or FIG. 9-FIG. 10. The first visual stimulus 406 can be presented to the first eye by a binocular display device, such as a first near-eye display of a binocular XR headset (e.g., glasses 200).

According to some examples, the method 1400 includes presenting a second visual stimulus 408 to a user's second eye (e.g., the other of the left eye 502 or right eye 504) at operation 1404. As described above, the second visual stimulus 408 is presented without the first modulation. The second visual stimulus 408 can be presented to the second eye by a binocular display device, such as a second near-eye display of a binocular XR headset (e.g., glasses 200).

Jointly, the first visual stimulus 406 and second visual stimulus 408 provide a dichoptic visual stimulus to the user's visual system. The first visual stimulus 406 and second visual stimulus 408 are configured to cause the user to perceive the first visual stimulus 406 and the second visual stimulus 408 as a single visual element (as described above) when the first eye is focused on the first visual stimulus 406 and the second eye is focused on the second visual stimulus 408. As described above, the first visual stimulus 406 and second visual stimulus 408 may be configured to cause the user to perceive the visual element as being unmodulated due to masking of the first modulation of the first visual stimulus 406 by one or more unmodulated portions of the second visual stimulus (e.g., in some examples, the static HSF features of the second visual stimulus 408 may mask the modulation of the HSF features of the first visual stimulus 406).

In some examples, multiple interactive visual elements can be presented as parts of a GUI. Each interactive visual element can be presented via the binocular display device as a distinct dichoptic visual stimulus having its own distinct characteristic modulation applied to its respective first visual stimulus 406. Thus, the GUI may include multiple dichoptic visual stimuli, each of which corresponds to a GUI element which can be selected or otherwise interacted with by a user by the user focusing his or her visual gaze on the given visual element.

In some examples, the visual element corresponding to the dichoptic visual stimulus is a virtual object (e.g., a 3D or 2D object perceived as occupying a position in 3D space) perceived stereoscopically by the first eye and second eye.

According to some examples, the method 1400 includes obtaining neural signals (such as EEG signals) from a neural signal capture device configured to detect the user's neural activity (such as neural signal capture device 703) at operation 1406.

As described above, in some cases, a user may need to close the second eye (viewing the second visual stimulus 408) in order to reliably trigger the BCI to detect that the first eye is focused on the first visual stimulus 406. Thus, in some cases, the neural signal capture device captures the neural signals at operation 1406 while the user is closing the second eye.

According to some examples, the method 1400 includes determining whether the user's first eye is focused on the first visual stimulus 406 at operation 1408. Because of the configuration of the visual stimuli, this generally means that the dichoptic visual stimulus is the object of visual focus, which is true if the first eye is focused on the first visual stimulus 406 and the second eye is focused on the second visual stimulus 408.

In some examples, the neural signals are processed to determine a strength of components of the neural signals having a property associated with the first modulation, as described above with reference to FIG. 6 through FIG. 12. For example, the first modulation may be applied with a characteristic frequency or duty cycle, and this characteristic frequency or duty cycle may be discernible from the neural signals as processed by the processing device 704.

According to some examples, the method 1400 includes causing a computing system (such as computer 220 or machine 1300) to execute a command associated with the dichoptic visual stimulus at operation 1410. In some examples, each of multiple dichoptic visual stimuli may be presented as GUI elements of a GUI via a binocular display device, and each dichoptic visual stimulus may be associated with a corresponding command. When the BCI determines, at operation 1408, that the user's gaze is focused on a given dichoptic visual stimulus, the computing system using the BCI for input executes the respective command associated with the given dichoptic visual stimulus. The command can include multiple operations in some cases. For example, a command associated with a given dichoptic visual stimulus may cause the computing system to first place the given dichoptic visual stimulus into an “selected” state, which may include changing the display of the dichoptic visual stimulus (e.g., changing its color, size, shape, or texture) to provide visual feedback to the user confirming that the user has selected the dichoptic visual stimulus using the BCI. The command may then include waiting for a predetermined dwell time while the method 1400 continues to loop through operation 1402 through operation 1408. The command can further include, if the user's visual attention is determined to have remained on the dichoptic visual stimulus throughout the dwell time, placing the dichoptic visual stimulus into an “activated” state (potentially with a change in visual appearance), and executing some further command associated with the given dichoptic visual stimulus. It will be appreciated that various behaviors, methods, algorithms, or operations can be triggered or controlled by the command executed by the computing system at operation 1410.

### Glossary

“Extended reality” (XR) refers, for example, to an interactive experience of a real-world environment where physical objects that reside in the real-world are “augmented” or enhanced by computer-generated digital content (also referred to as virtual content or synthetic content). XR can also refer to a system that enables a combination of real and virtual worlds, real-time interaction, and 3D registration of virtual and real objects. A user of an XR system perceives virtual content that appears to be attached to, or interacts with, a real-world physical object.

“Client device” refers, for example, to any machine that interfaces to a communications network to obtain resources from one or more server systems or other client devices. A client device may be, but is not limited to, a mobile phone, desktop computer, laptop, portable digital assistants (PDAs), smartphones, tablets, ultrabooks, netbooks, laptops, multi-processor systems, microprocessor-based or programmable consumer electronics, game consoles, set-top boxes, or any other communication device that a user may use to access a network.

“Communication network” refers, for example, to one or more portions of a network that may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network, and the coupling may be a Code Division Multiple Access (CDMA) connection, a Global System for Mobile communications (GSM) connection, or other types of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth-generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long-range protocols, or other data transfer technology.

“Component” refers, for example, to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various examples, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein. A hardware component may also be implemented mechanically, electronically, or any suitable combination thereof. For example, a hardware component may include dedicated circuitry or logic that is permanently configured to perform certain operations. A hardware component may be a special-purpose processor, such as a field-programmable gate array (FPGA) or an application-specific integrated circuit (ASIC). A hardware component may also include programmable logic or circuitry that is temporarily configured by software to perform certain operations. For example, a hardware component may include software executed by a general-purpose processor or other programmable processors. Once configured by such software, hardware components become specific machines (or specific components of a machine) uniquely tailored to perform the configured functions and are no longer general-purpose processors. It will be appreciated that the decision to implement a hardware component mechanically, in dedicated and permanently configured circuitry, or in temporarily configured circuitry (e.g., configured by software), may be driven by cost and time considerations. Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering examples in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time. Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In examples in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors or processor-implemented components. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an API). The performance of certain of the operations may be distributed among the processors, not only residing within a single machine, but deployed across a number of machines. In some examples, the processors or processor-implemented components may be located in a single geographic location (e.g., within a home environment, an office environment, or a server farm). In other examples, the processors or processor-implemented components may be distributed across a number of geographic locations.

“Computer-readable storage medium” refers, for example, to both machine-storage media and transmission media. Thus, the terms include both storage devices/media and carrier waves/modulated data signals. The terms “machine-readable medium,” “computer-readable medium” and “device-readable medium” mean the same thing and may be used interchangeably in this disclosure.

“Machine storage medium” refers, for example, to a single or multiple storage devices and media (e.g., a centralized or distributed database, and associated caches and servers) that store executable instructions, routines and data. The term shall accordingly be taken to include, but not be limited to, solid-state memories, and optical and magnetic media, including memory internal or external to processors. Specific examples of machine-storage media, computer-storage media and device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks The terms “machine-storage medium,” “device-storage medium,” “computer-storage medium” mean the same thing and may be used interchangeably in this disclosure. The terms “machine-storage media,” “computer-storage media,” and “device-storage media” specifically exclude carrier waves, modulated data signals, and other such media, at least some of which are covered under the term “signal medium.”

“Non-transitory computer-readable storage medium” refers, for example, to a tangible medium that is capable of storing, encoding, or carrying the instructions for execution by a machine.

“Signal medium” refers, for example, to any intangible medium that is capable of storing, encoding, or carrying the instructions for execution by a machine and includes digital or analog communications signals or other intangible media to facilitate communication of software or data. The term “signal medium” shall be taken to include any form of a modulated data signal, carrier wave, and so forth. The term “modulated data signal” means a signal that has one or more of its characteristics set or changed in such a matter as to encode information in the signal. The terms “transmission medium” and “signal medium” mean the same thing and may be used interchangeably in this disclosure.

“Stereoscopic vision” refers to how the human visual system uses the differences between the images seen by each eye to figure out how far away objects are and their three-dimensional shapes and orientations. It is generally believed that noticing these side-to-side differences is what allows humans to perceive depth.

“User device” refers, for example, to a device accessed, controlled or owned by a user and with which the user interacts perform an action, or an interaction with other users or computer systems.

### EXAMPLES

To better illustrate the systems and methods disclosed herein, a non-limiting list of examples is provided here:

Example 1 is a method, comprising: presenting a dichoptic visual stimulus to a user's eyes by: presenting a first visual stimulus to a first eye of the user, the first visual stimulus being presented with a first modulation; and presenting a second visual stimulus to a second eye of the user, the second visual stimulus being presented without the first modulation, the first visual stimulus and second visual stimulus being configured to: cause the user to perceive the first visual stimulus and the second visual stimulus as a single visual element when the first eye is focused on the first visual stimulus and the second eye is focused on the second visual stimulus; and cause the user to perceive the visual element as being unmodulated by the first modulation due to masking of the first modulation of the first visual stimulus by one or more portions of the second visual stimulus presented without the first modulation; obtaining neural signals from a neural signal capture device configured to detect neural activity of the user; and in response to determining, based on the neural signals, that the first eye is focused on the first visual stimulus, causing a computing system to execute a command associated with the dichoptic visual stimulus.

In Example 2, the subject matter of Example 1 includes, wherein: the determining that the user's first eye is focused on the first visual stimulus comprises: determining a strength of components of the neural signals having a property associated with the first modulation.

In Example 3, the subject matter of Examples 1-2 includes, wherein: the visual element is a virtual object perceived stereoscopically by the first eye and second eye.

In Example 4, the subject matter of Examples 1-3 includes, wherein: the first modulation comprises temporal modulation whereby the first visual stimulus is presented in at least two different states at each of at least two different times.

In Example 5, the subject matter of Example 4 includes, wherein: the temporal modulation comprises cyclic sequential presentation of the at least two different states of the first visual stimulus at a cyclic frequency of at least 6 Hz.

In Example 6, the subject matter of Examples 4-5 includes, wherein: the at least two different states comprises: a first state comprising a first view of the visual element; and a second state comprising the first view of the visual element, excluding one or more high spatial frequency visual features of the visual element; and the second visual stimulus comprises a second view of the visual element.

In Example 7, the subject matter of Examples 4-6 includes, wherein: the at least two different states comprises: a first state comprising a first view of the visual element; and a second state comprising the first view of the visual element, overlaid with a visual texture or pattern; and the second visual stimulus comprises a second view of the visual element, overlaid with the visual texture or pattern.

In Example 8, the subject matter of Examples 4-7 includes, wherein: the at least two different states comprises: a first state comprising a first view of the visual element, overlaid with a first visual texture or pattern; and a second state comprising the first view of the visual element, overlaid with a second visual texture or pattern; and the second visual stimulus comprises a second view of the visual element, overlaid with the first visual texture or pattern.

In Example 9, the subject matter of Examples 4-8 includes, wherein: the second visual stimulus is presented with a second temporal modulation, different from the first modulation, whereby the second visual stimulus is presented in at least two different states at each of at least two different times.

In Example 10, the subject matter of Examples 4-9 includes, wherein: the at least two different states comprises: a first state comprising a first view of the visual element; and a second state comprising the first view of the visual element, at least a portion of the visual element being modified with respect to at least one of contrast or luminance; and the second visual stimulus comprises a second view of the visual element.

In Example 11, the subject matter of Examples 1-10 includes, wherein: the neural signal capture device captures the neural signals while the user is closing the second eye.

Example 12 is a computing system, comprising: at least one display device; a neural signal capture device configured to detect neural activity of a user; one or more processors; and a memory storing instructions that, when executed by the one or more processors, cause the computing system to perform operations comprising: presenting a dichoptic visual stimulus to a user's eyes by: presenting a first visual stimulus to a first eye of the user via the at least one display device, the first visual stimulus being presented with a first modulation; and presenting a second visual stimulus to a second eye of the user via the at least one display device, the second visual stimulus being presented without the first modulation, the first visual stimulus and second visual stimulus being configured to: cause the user to perceive the first visual stimulus and the second visual stimulus as a single visual element when the first eye is focused on the first visual stimulus and the second eye is focused on the second visual stimulus; and cause the user to perceive the visual element as being unmodulated by the first modulation due to masking of the first modulation of the first visual stimulus by one or more portions of the second visual stimulus presented without the first modulation; obtaining neural signals from the neural signal capture device; and in response to determining, based on the neural signals, that the first eye is focused on the first visual stimulus, executing a command associated with the dichoptic visual stimulus.

In Example 13, the subject matter of Example 12 includes, wherein: the determining that the user's first eye is focused on the first visual stimulus comprises: determining a strength of components of the neural signals having a property associated with the first modulation.

In Example 14, the subject matter of Examples 12-13 includes, wherein: the visual element is a virtual object perceived stereoscopically by the first eye and second eye.

In Example 15, the subject matter of Examples 12-14 includes, wherein: the first modulation comprises temporal modulation whereby the first visual stimulus is presented in at least two different states at each of at least two different times.

In Example 16, the subject matter of Example 15 includes, wherein: the temporal modulation comprises cyclic sequential presentation of the at least two different states of the first visual stimulus at a cyclic frequency of at least 6 Hz.

In Example 17, the subject matter of Examples 15-16 includes, wherein: the at least two different states comprises: a first state comprising a first view of the visual element; and a second state comprising the first view of the visual element, excluding one or more high spatial frequency visual features of the visual element; and the second visual stimulus comprises a second view of the visual element.

In Example 18, the subject matter of Examples 15-17 includes, wherein: the at least two different states comprises: a first state comprising a first view of the visual element; and a second state comprising the first view of the visual element, overlaid with a visual texture or pattern; and the second visual stimulus comprises a second view of the visual element, overlaid with the visual texture or pattern.

In Example 19, the subject matter of Examples 15-18 includes, wherein: the at least two different states comprises: a first state comprising a first view of the visual element; and a second state comprising the first view of the visual element, at least a portion of the visual element being modified with respect to at least one of contrast or luminance; and the second visual stimulus comprises a second view of the visual element.

Example 20 is a non-transitory computer-readable storage medium storing instructions that, when executed by one or more processors of a computing system, cause the computing system to perform operations comprising: presenting a dichoptic visual stimulus to a user's eyes by: presenting a first visual stimulus to a first eye of the user, the first visual stimulus being presented with a first modulation; and presenting a second visual stimulus to a second eye of the user, the second visual stimulus being presented without the first modulation, the first visual stimulus and second visual stimulus being configured to: cause the user to perceive the first visual stimulus and the second visual stimulus as a single visual element when the first eye is focused on the first visual stimulus and the second eye is focused on the second visual stimulus; and cause the user to perceive the visual element as being unmodulated by the first modulation due to masking of the first modulation of the first visual stimulus by one or more portions of the second visual stimulus presented without the first modulation; obtaining neural signals from a neural signal capture device configured to detect neural activity of the user; and in response to determining, based on the neural signals, that the first eye is focused on the first visual stimulus, executing a command associated with the dichoptic visual stimulus.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20. Example 23 is a system to implement of any of Examples 1-20.

Example 24 is a method to implement of any of Examples 1-20.

Further particular and preferred aspects of the present disclosure are set out in the accompanying independent and dependent claims. It will be appreciated that features of the dependent claims may be combined with features of the independent claims in combinations other than those explicitly set out in the claims.

1. A method, comprising:
presenting a dichoptic visual stimulus to a user's eyes by:
presenting a first visual stimulus to a first eye of the user, the first visual stimulus being presented with a first modulation; and
presenting a second visual stimulus to a second eye of the user, the second visual stimulus being presented without the first modulation,
the first visual stimulus and second visual stimulus being configured to:
cause the user to perceive the first visual stimulus and the second visual stimulus as a single visual element when the first eye is focused on the first visual stimulus and the second eye is focused on the second visual stimulus; and
cause the user to perceive the visual element as being unmodulated by the first modulation due to masking of the first modulation of the first visual stimulus by one or more portions of the second visual stimulus presented without the first modulation;
obtaining neural signals from a neural signal capture device configured to detect neural activity of the user; and
in response to determining, based on the neural signals, that the first eye is focused on the first visual stimulus, causing a computing system to execute a command associated with the dichoptic visual stimulus.

2. The method of claim 1, wherein:
the determining that the user's first eye is focused on the first visual stimulus comprises:
determining a strength of components of the neural signals having a property associated with the first modulation.

3. The method of claim 1, wherein:
the visual element is a virtual object perceived stereoscopically by the first eye and second eye.

4. The method of claim 1, wherein:
the first modulation comprises temporal modulation whereby the first visual stimulus is presented in at least two different states at each of at least two different times.

5. The method of claim 4, wherein:
the temporal modulation comprises cyclic sequential presentation of the at least two different states of the first visual stimulus at a cyclic frequency of at least 6 Hz.

6. The method of claim 4, wherein:
the at least two different states comprises:
a first state comprising a first view of the visual element; and
a second state comprising the first view of the visual element, excluding one or more high spatial frequency visual features of the visual element; and
the second visual stimulus comprises a second view of the visual element.

7. The method of claim 4, wherein:
the at least two different states comprises:
a first state comprising a first view of the visual element; and
a second state comprising the first view of the visual element, overlaid with a visual texture or pattern; and
the second visual stimulus comprises a second view of the visual element, overlaid with the visual texture or pattern.

8. The method of claim 4, wherein:
the at least two different states comprises:
a first state comprising a first view of the visual element, overlaid with a first visual texture or pattern; and
a second state comprising the first view of the visual element, overlaid with a second visual texture or pattern; and
the second visual stimulus comprises a second view of the visual element, overlaid with the first visual texture or pattern.

9. The method of claim 4, wherein:
the second visual stimulus is presented with a second temporal modulation, different from the first modulation, whereby the second visual stimulus is presented in at least two different states at each of at least two different times.

10. The method of claim 4, wherein:
the at least two different states comprises:
a first state comprising a first view of the visual element; and
a second state comprising the first view of the visual element, at least a portion of the visual element being modified with respect to at least one of contrast or luminance; and
the second visual stimulus comprises a second view of the visual element.

11. The method of claim 1, wherein:
the neural signal capture device captures the neural signals while the user is closing the second eye.

12. A computing system, comprising:
at least one display device;
a neural signal capture device configured to detect neural activity of a user;
one or more processors; and
a memory storing instructions that, when executed by the one or more processors, cause the computing system to perform operations comprising:
presenting a dichoptic visual stimulus to a user's eyes by:
presenting a first visual stimulus to a first eye of the user via the at least one display device, the first visual stimulus being presented with a first modulation; and
presenting a second visual stimulus to a second eye of the user via the at least one display device, the second visual stimulus being presented without the first modulation,
the first visual stimulus and second visual stimulus being configured to:
cause the user to perceive the first visual stimulus and the second visual stimulus as a single visual element when the first eye is focused on the first visual stimulus and the second eye is focused on the second visual stimulus; and
cause the user to perceive the visual element as being unmodulated by the first modulation due to masking of the first modulation of the first visual stimulus by one or more portions of the second visual stimulus presented without the first modulation;
obtaining neural signals from the neural signal capture device; and
in response to determining, based on the neural signals, that the first eye is focused on the first visual stimulus, executing a command associated with the dichoptic visual stimulus.

13. The computing system of claim 12, wherein:
the determining that the user's first eye is focused on the first visual stimulus comprises:
determining a strength of components of the neural signals having a property associated with the first modulation.

14. The computing system of claim 12, wherein:
the visual element is a virtual object perceived stereoscopically by the first eye and second eye.

15. The computing system of claim 12, wherein:
the first modulation comprises temporal modulation whereby the first visual stimulus is presented in at least two different states at each of at least two different times.

16. The computing system of claim 15, wherein:
the temporal modulation comprises cyclic sequential presentation of the at least two different states of the first visual stimulus at a cyclic frequency of at least 6 Hz.

17. The computing system of claim 15, wherein:
the at least two different states comprises:
a first state comprising a first view of the visual element; and
a second state comprising the first view of the visual element, excluding one or more high spatial frequency visual features of the visual element; and
the second visual stimulus comprises a second view of the visual element.

18. The computing system of claim 15, wherein:
the at least two different states comprises:
a first state comprising a first view of the visual element; and
a second state comprising the first view of the visual element, overlaid with a visual texture or pattern; and
the second visual stimulus comprises a second view of the visual element, overlaid with the visual texture or pattern.

19. The computing system of claim 15, wherein:
the at least two different states comprises:
a first state comprising a first view of the visual element; and
a second state comprising the first view of the visual element, at least a portion of the visual element being modified with respect to at least one of contrast or luminance; and
the second visual stimulus comprises a second view of the visual element.

20. A non-transitory computer-readable storage medium storing instructions that, when executed by one or more processors of a computing system, cause the computing system to perform operations comprising:
presenting a dichoptic visual stimulus to a user's eyes by:
presenting a first visual stimulus to a first eye of the user, the first visual stimulus being presented with a first modulation; and
presenting a second visual stimulus to a second eye of the user, the second visual stimulus being presented without the first modulation,
the first visual stimulus and second visual stimulus being configured to:
cause the user to perceive the first visual stimulus and the second visual stimulus as a single visual element when the first eye is focused on the first visual stimulus and the second eye is focused on the second visual stimulus; and
cause the user to perceive the visual element as being unmodulated by the first modulation due to masking of the first modulation of the first visual stimulus by one or more portions of the second visual stimulus presented without the first modulation;
obtaining neural signals from a neural signal capture device configured to detect neural activity of the user; and
in response to determining, based on the neural signals, that the first eye is focused on the first visual stimulus, executing a command associated with the dichoptic visual stimulus.
